(19) United States
(12) Patent Application Publication
(10) Pub. No.: US 2017/0020890 A1
(43) Pub. Date: Jan. 26, 2017

(54) PHARMACEUTICAL COMPOSITION COMPRISING FATTY ACID DERIVATIVE

(71) Applicants: SUCAMPO AG, Zug (CH); R-TECH UENO, LTD., Tokyo-to (JP)

(72) Inventors: Ryuji UENO, Easton, MD (US); Ryu HIRATA, Hyogo (JP); Yasuhiro HARADA, Tokyo-to (JP); Tomoko TSUDA, Tokyo-to (JP)

(21) Appl. No.: 15/184,474

(22) Filed: Jun. 16, 2016

Related U.S. Application Data
(60) Provisional application No. 62/182,084, filed on Jun. 19, 2015.

(51) Int. Cl. A61K 31/558; A61K 47/18; A61K 47/14; A61K 9/00

(52) U.S. Cl. CPC A61K 31/558; A61K 9/0053; A61K 47/183; A61K 47/14

(57) ABSTRACT

An oral pharmaceutical composition comprising (a) a specific fatty acid derivative, (b) a specific sweetening agent and (c) a pharmaceutically acceptable oily vehicle, and an oily liquid formulation comprising thereof is provided.

### CROSS-REFERENCE TO RELATED APPLICATIONS

This application claims priority to U.S. Provisional Patent Application No. 62/182,084, filed Jun. 19, 2015, which is incorporated herein by reference.

### TECHNICAL FIELD

The present invention relates to an oral pharmaceutical composition comprising (a) a specific fatty acid derivative, (b) a specific sweetening agent and (c) a pharmaceutically acceptable oily vehicle, and an oily liquid formulation comprising thereof.

### BACKGROUND

Oral administration of pharmaceuticals is one of the most popular methods of drug delivery system. The common oral dosage form include, liquid formulation like solution, suspension and emulsion, solid dosage form like tablet, capsule and liquid filled capsule etc. However patient such as children and the elderly, often experience difficulty in swallowing solid oral dosage form, for these patients the drug are mostly in liquid dosage form such as solution, suspension and emulsion. This dosage form usually lead to perceptible exposure of active ingredients to taste buds if the active ingredient is bitter this gives extremely unpleasant bitter taste. Further, in the case of oily vehicle in the liquid formulation, the oily vehicle itself can be a factor of the unpleasant taste in the formulation. Accordingly, masking of unpleasant taste characteristics of drug is an important factor in formulation of these agents.

Fatty acid derivatives are members of class of organic carboxylic acids, which are contained in tissues or organs of human or other mammals, and exhibit a wide range of physiological activity. Some fatty acid derivatives found in nature generally have a prostanoic acid skeleton as shown in the formula (A):

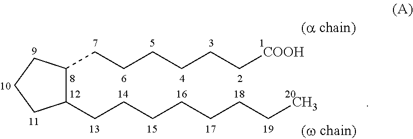 [SMILES: CCCCCCCCC1CCC[C@@H]1CCCCCCC(=O)O]

On the other hand, some of synthetic prostaglandin (PG) analogues have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of β-configuration).

PGs are known to have various pharmacological and physiological activities, for example, vasodilatation, inducing of inflammation, platelet aggregation, stimulating uterine muscle, stimulating intestinal muscle, anti-ulcer effect and the like.

Prostones, having an oxo group at position 15 of prostanoic acid skeleton (15-keto type) and having a single bond between positions 13 and 14 and an oxo group at position 15 (13,14-dihydro-15-keto type), are fatty acid derivatives known as substances naturally produced by enzymatic actions during metabolism of the primary PGs and have some therapeutic effect. Prostones have been disclosed in U.S. Pat. Nos. 5,073,569, 5,534,547, 5,225,439, 5,166,174, 5,428,062 5,380,709 5,886,034 6,265,440, 5,106,869, 5,221,763, 5,591,887, 5,770,759 and 5,739,161, the contents of these references are herein incorporated by reference.

As one of commercially available pharmaceutical product for prostones, Amitiza® (lubiprostone) capsule is indicated for the treatment of chronic idiopathic constipation (CIC) and opioid-induced constipation (OIC) with chronic, non-cancer pain. Lubiprostone is also indicated for irritable bowel syndrome with constipation (IBS-C) in women.

A sweetening agent can play a number of important roles in solid and oral liquid formulations such as enhancing flavor, masking bitter taste and increasing viscosity. The search for the perfect sweetening agent continues, but it has long been recognized that the ideal sweetening agent does not exist.

### DISCLOSURE OF THE INVENTION

The present invention relates to an oral pharmaceutical composition comprising:

(a) a fatty acid derivative represented by the formula (I):

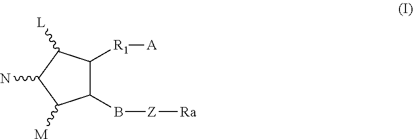

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

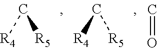

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur,

(b) a sweetening agent selected from the group consisting of neotame, saccharin, sucralose, and a mixture thereof, and (c) a pharmaceutically acceptable oily vehicle.

The present invention further relates to an oily liquid formulation comprising (a) a fatty acid derivative represented by the formula described above, (b) a sweetening agent selected from the group consisting of neotame, saccharin, sucralose, and a mixture thereof, and (c) a pharmaceutically acceptable oily vehicle.

### DETAILED DESCRIPTION OF THE INVENTION

### (a) Fatty Acid Derivative

The nomenclature of the fatty acid derivative used herein is based on the numbering system of the prostanoic acid represented in the above formula (A).

The formula (A) shows a basic skeleton of the C-20 fatty acid derivative, but the present invention is not limited to those having the same number of carbon atoms. In the formula (A), the numbering of the carbon atoms which constitute the basic skeleton of the fatty acid derivatives starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C-1). Similarly, when the number of carbon atoms is decreased in the ω-chain, the number is deleted in the order starting from position 20; and when the number of carbon atoms is increased in the ω-chain, the carbon atoms at the position 21 or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-11-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-carboxybutyl)-PG compound. Further, a fatty acid derivative whose w-chain is extended by two carbon atoms, that is, having 10 carbon atoms in the w-chain is named as 20-ethyl-PG compound. These compounds, however, may also be named according to the IUPAC nomenclatures.

Examples of the analogues including substitution compounds or derivatives of the above described fatty acid derivative include a fatty acid derivative whose carboxy group at the end of the alpha chain is esterified; a fatty acid derivative whose α chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a fatty acid derivative having a lower alkyl or a hydroxy (lower) alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have a w chain shorter than that of the primary PGs and a substituent such as alkoxy, cycloalkyl, cycloalkyloxy, phenoxy and phenyl at the end of the truncated ω-chain.

A fatty acid derivative used in the present invention is represented by the formula (I):

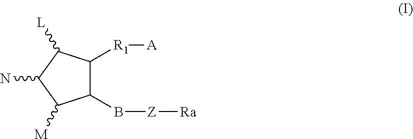

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

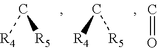

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

A preferred compound used in the present invention is represented by the formula (II):

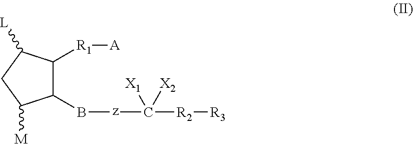

wherein L and M are hydrogen atom, hydroxy, halogen, lower alkyl, hydroxy(lower)alkyl, lower alkanoyloxy or oxo, wherein at least one of L and M is a group other than hydrogen, and the five-membered ring may have one or more double bonds;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

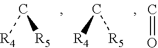

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;

X₁ and X₂ are hydrogen, lower alkyl, or halogen;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur;

R₂ is a single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group, and at least one of carbon atom in the aliphatic hydrocarbon is optionally substituted by oxygen, nitrogen or sulfur.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein He is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino) ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo, and especially, L and M are both hydroxy, or L is oxo and M is hydrogen or hydroxy.

Preferred example of A is —COOH, its pharmaceutically acceptable salt, ester or amide thereof.

Preferred example of X₁ and X₂ are both being halogen atoms, and more preferably, fluorine atoms, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur.

Examples of R₁ include, for example, the following groups:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom. Further, at least one carbon atom in the aliphatic hydrocarbon is optionally substituted by oxygen, nitrogen or sulfur.

Preferable compounds include Ra is substituted by halogen and/or Z is C═O in the formula (I), or one of X1 and X2 is substituted by halogen and/or Z is C═O in the formula (II).

Example of the preferred embodiment is a (−)-7-[(2R,4aR,5R,7aR)-2-(1,1-difluoropentyl)-2-hydroxy-6-oxooctahydrocyclopenta[b]pyran-5-yl]heptanoic acid (lubiprostone) or (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid (cobiprostone), (−)-7-[(1R,2R)-2-(4,4-difluoro-3-oxooctyl)-5-oxocyclopentyl]heptanoic acid, (E)-7-[(1R,2R)-2-(4,4-difluoro-3-oxooctyl)-5-oxocyclopentyl]hept-2-enoic acid, an isomer (including tautomeric isomer) thereof and a functional derivative thereof.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, the fatty acid derivative which is dihydro between 13 and 14, and keto(═O) at 15 position may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the present invention includes both isomers.

Further, the fatty acid derivatives used in the invention include the bicyclic compound and analogs or derivatives thereof.

The bicyclic compound is represented by the formula (III)

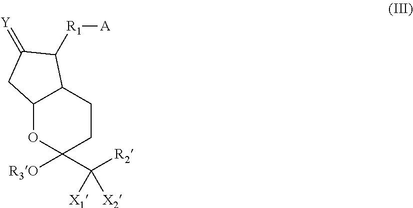

wherein, A is —CH₃, or —CH₂OH, —COCH₂OH, —COOH or a functional derivative thereof;

X₁′ and X₂′ are hydrogen, lower alkyl, or halogen;

Y is

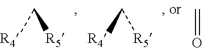

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated divalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

R₃′ is hydrogen, lower alkyl, cyclo(lower)alkyl, aryl or heterocyclic group.

Furthermore, while the compounds used in the invention may be represented by a formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485 (these cited references are herein incorporated by reference).

### (b) Sweetening Agent

Neotame is an artificial sweetening agent having IUPAC name as 3S)-3-(3,3-Dimethylbutylamino)-4-[[(2S)-1-methoxy-1-oxo-3-phenylpropan-2-yl]amino]-4-oxobutanoic acid with the following chemical structure.

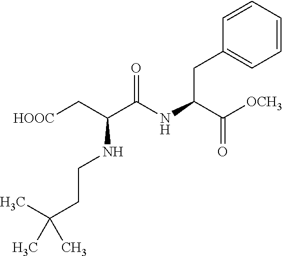 [SMILES: COC(=O)[C@H](Cc1ccccc1)NC(=O)[C@H](CC(=O)O)NCCC(C)(C)C]

Saccharin is an artificial sweetening agent having IUPAC name as 2H-1λ⁶, 2-benzothiazol-1,1,3-trione (other name is Benzoic sulfimide) with the following chemical structure.

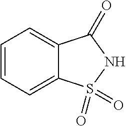 [SMILES: O=C1NS(=O)(=O)c2ccccc21]

Sucralose is an artificial sweetening agent, having IUPAC name as 1,6-Dichloro-1,6-dideoxy-β-D-fructofuranosyl-4-chloro-4-deoxy-α-D-galactopyranoside (other name is 1′,4,6′-trichlorogalactosucrose) with the following chemical structure.

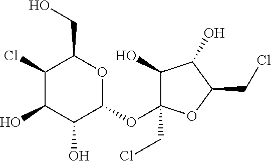 [SMILES: OC[C@H]1O[C@H](O[C@]2(CCl)O[C@H](CCl)[C@@H](O)[C@@H]2O)[C@H](O)[C@@H](O)[C@H]1Cl]

### (c) Pharmaceutically Acceptable Oily Vehicle

According to this embodiment, a pharmaceutically acceptable oily vehicle is not specifically limited as long as the vehicle can dissolve or disperse the fatty acid derivative of the present invention therein and does not significantly deteriorate the stability of the compound.

Examples of the pharmaceutically acceptable vehicles may include, but not limited to, fatty acid esters, i.e. an ester of fatty acid and an alcohol, and polyols.

Examples of the fatty acid esters used in the present invention may include fatty acid esters obtained from a fatty acid and an alcohol, for example, saturated or unsaturated glycerides which may have a branched chain.

Preferred fatty acid esters may include a medium or higher chain fatty acid having at least C6, preferably C6-24 carbon atoms, for example caproic acid (C6), caprylic acid (C8), capric acid (C10), lauric acid (C12), myristic acid (C14), palmitic acid (C16), palmitoleic acid (C16), stearic acid (C18), oleic acid (C18), linoleic acid (C18), linolenic acid (C18), ricinolic acid (C18) and arachic acid (C20). Preferred alcohols which consists the fatty acid ester may comprise C1-6 primary alcohol and polyols such as glycerin, polyethyleneglycol and propyleneglycol.

Preferred fatty acid esters may include a glyceride or a propylene glycol ester of a saturated or unsaturated fatty acid which may have a branched chain. Two or more glycerides may be used as a mixture.

Examples of the mixture of glycerides are mixture of caprylic acid triglyceride and capric acid triglyceride, vegetable oils such as castor oil, corn oil, olive oil, sesame oil, rape oil, salad oil, cottonseed oil, camellia oil, peanut oil, palm oil and sunflower oil

A fatty acid ester derived from a fatty acid and a monovalent alcohol is also preferably used as a pharmaceutically acceptable vehicle. The fatty acid ester may preferably be an ester of C8-20 fatty acid and a C2-3 monovalent alcohol, such as isopropyl myristate, isopropyl palmitate, ethyl linoleate and ethyl oleate.

Examples of polyols may preferably include alcohols having two or three hydroxy groups such as glycerin, polyethylene glycol and propylene glycol.

Examples of other oil solvent other than the fatty acid ester includes, but not limited to, mineral oil, liquid paraffin, and tocopherol.

The dose may vary depending on the strain of the animal, age, body weight, symptom to be treated, desired therapeutic effect, administration route, term of treatment and the like. A satisfactory effect can be obtained by systemic administration 1-4 times per day or continuous administration at the amount of 0.00001-500 mg/kg per day, more preferably 0.0001-100 mg/kg.

The compound may preferably be formulated in a pharmaceutical composition suitable for administration in a conventional manner.

The composition of the present invention may further contain physiologically acceptable additives. Said additives may include the ingredients used with the present compounds such as excipient, diluent, filler, resolvent, lubricant, adjuvant, binder, disintegrator, coating agent, aerozoling agent, emulsifier, dispersing agent, suspending agent, thickener, tonicity agent, buffering agent, soothing agent, preservative, antioxidant, corrigent, flavor, colorant, a functional material such as cyclodextrin and biodegradable polymer, stabilizer. The additives are well known to the art and may be selected from those described in general reference books of pharmaceutics.

According to the present invention, the composition further comprises (d) a flavor such as vanilla.

The amount of the above-defined compound in the composition of the invention may vary depending on the formulation of the composition, and may generally be 0.000001-10.0%, more preferably 0.00001-5.0%, most preferably 0.0001-1%.

Examples of liquid compositions for oral administration include emulsions, solutions, suspensions, syrups and elixirs and the like. Said composition may further contain a conventionally used inactive diluents e.g. purified water or ethyl alcohol. The composition may contain additives other than the inactive diluents such as adjuvant e.g. wetting agents and suspending agents, flavors, fragrance and preservatives.

The composition of the present invention may be in the form of spraying composition, which contains one or more active ingredients and may be prepared according to a known method.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization.

According to the present invention, the oral pharmaceutical composition is useful for the oily liquid formulation like solution, suspension and emulsion with improving the taste and/or retaining the stability of the composition. Especially, the composition of the present invention is useful for the children and the elderly, often experience difficulty in swallowing solid oral dosage form, for these patients the drug are mostly in liquid dosage form such as solution, suspension and emulsion.

The pharmaceutical composition of the present invention may contain a single active ingredient or a combination of two or more active ingredients, as far as they are not contrary to the objects of the present invention.

In a combination of plural active ingredients, their respective contents may be suitably increased or decreased in consideration of their therapeutic effects and safety.

The term “combination” used herein means two or more active ingredient are administered to a patient simultaneously in the form of a single entity or dosage, or are both administered to a patient as separate entities either simultaneously or sequentially with no specific time limits, wherein such administration provides therapeutically effective levels of the two components in the body, preferably at the same time.

The present invention will be described in detail with reference to the following example, which, however, is not intended to limit the scope of the present invention.

### Example 1

18 mg of neotame, 20 mg of saccharin, 1 mg of sodium saccharin or 5 mg of thaumatin was mixed with medium-chain triglycerides (MCT; USP/NF) to give the total weight of 180 g, 20 g, 100 g or 500 g respectively.

The solubility and sensory test of neotame, saccharin, sodium saccharin or thaumatin is shown in Table 1.

TABLE 1

| | Solubility to | Sensory test |
|---|---|---|
| Sweetening agent | MCT | (sweetness) |
| Neotame | 0.01% | + |
| Saccharin | 0.1% | + |
| Sodium saccharin | <0.001% | − |
| Thaumatin | <0.001% | − |

The data indicated that neotame and saccharin solved in MCT as well as showing the desired sweetness (+), whereas sodium saccharin and thaumatin did not solve in MCT nor show the desired sweetness (−)

### Example 2

Compound 1 ((−)-7-[(2R,4aR,5R,7aR)-2-(1,1-difluoro pentyl)-2-hydroxy-6-oxooctahydrocyclopenta[b]pyran-5-yl] heptanoic acid) was dissolved in medium-chain triglycerides (USP/NF) to give 240 μg/mL solution. Sweetening agent/flavor shown in table 2 was added to the solution. The precise amount of Compound 1 in the solution was determined by means of HPLC (day 0). Then, the solution was put in a High Density Polyethylene container and kept at 55° C. for 1 month, and then the precise amount of the compound 1 was determined by means of HPLC (1 month).

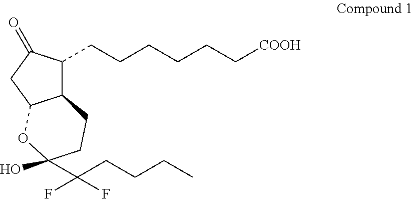 [SMILES: CCCCC(F)(F)[C@@]1(O)CC[C@H]2[C@@H](CC(=O)[C@@H]2CCCCCCC(=O)O)O1]

The determination of the amount of the compound 1 in a solution was carried as follows. About 0.5 mL of the sample was mixed with exactly 2 mL of internal standard solution and then with acetonitrile to give 25 mL of sample solution. About 24 mg of the reference standard of compound 1 was weighed precisely and mixed with acetonitrile to give exactly 100 mL solution. Exactly 2 mL of the solution was pipetted and mixed with acetonitrile to give 20 mL. Exactly 5 mL of the solution was pipetted and mixed with exactly 2 mL of the internal standard solution, and then mixed with acetonitrile to give 25 mL of standard solution.

The fluorescent labeling agent was added to the respective solution, stirred and stood at room temperature. Then, the respective solution in an amount that theoretically gives 3.6 ng of compound 1 was loaded on the column and analyzed under the condition as follows:

HPLC analysis condition:

Column: 5 mm×25 cm stainless steel column packed with octadecylsilanized silica gel for HPLC (5 μm)

Mobile phase: a mixture of ethyl acetate HPLC grade: methanol HPLC grade: ethanol HPLC grade: ammonium acetate (0.05 mol/L)

Temperature: 35° C.

Detector: spectrophotofluorometer

Results are shown in Table 2.

TABLE 2
Assay results of compound 1 after 55° C. storage

| | Sweetening agent/flavor | | | | |
|---|---|---|---|---|---|
| | Neotame | Saccharin | Vanilla | day 0¹⁾ | 1 month¹⁾ |
| 1 | 0.008% | — | 0.15% | 99.6% | 97.6% |
| 2 | — | 0.04% | 0.2% | 99.2% | 95.0% |
| 3 | — | — | 0.2% | 99.4% | 97.6% |

¹⁾percentage based on the theoretical amount (240 μg/mL)

### Example 3

### Seven Experienced Pharmaceutical Sensory Panelists were Screened and Enrolled in the Study

The throat catch, the primary challenge of the oral liquid formulation was studied for unflavored compound 1 with vehicle (MCT), vanilla flavored compound 1 with vehicle (MCT) same as formulation 3 in example 2, and vehicle (MCT) only.

Intensity of Throat catch was scored as follows.

### Intensity Scale: 0=None

1=Slight
2=Moderate
3=Strong

The results of the throat catch intensity 3 minutes after the administration were shown in Table 3.

TABLE 3

| Formulation | Throat Catch Intensity |
|---|---|
| Vehicle (MCT) only | ½ |
| Compound 1 + Vehicle (MCT) | 1½ |
| Compound 1 + Vehicle (MCT) + | 1½ |
| vanilla | |

The data indicated that Compound 1 is higher throat catch intensity than vehicle, and vanilla did not improve the throat catch caused by compound 1.

### Example 4

### Six Experienced Pharmaceutical Sensory Panelists were Enrolled in the Study

The quality of both the initial flavor and aftertaste were evaluated for formulation 1 (0.008% of neotame, 0.15% of Vanilla), formulation 2 (0.04% of Saccharin and 0.2% vanilla) and formulation 3 (0.2% of vanilla) described in Example 2.

The quality of the initial flavor and aftertaste were calculated as follows.

Initial Flavor Quality−measured directly by Amplitude*. The target Amplitude for oral pharmaceuticals is 1.5;

*Amplitude: Initial overall perception of the balance and fullness of a flavored product; considering the appropriateness of aromas and flavor notes present, their blend and intensity and existence of off-notes.

### Amplitude Scale: 0=None

1=Low
2=Moderate
3=High
   Aftertaste Flavor Quality—calculated as the sum of the intensity of off-notes and mouthfeels measured in the aftertaste, i.e., 1, 3, 5, 10, 15, 20, 25 and 30 minutes. A short aftertaste is desirable, which would be represented by lower summations for each attribute.

The results were shown in table 4.

TABLE 4

| | | Aftertaste Flavor | | |
|---|---|---|---|---|
| | | Quality | | |
| | Initial Flavor | (Sum of Attributes) | | |
| Compound 1 | Quality | | Throat | Tongue |
| Formulation | (Amplitude) | Bitter | Catch | Sting |
| Vanilla | 0.5-1 | 0.5 | 7.5 | 7 |
| Saccharin + Vanilla | 1 | 0.25 | 6.5 | 4.75 |
| Neotame + Vanilla | 1-1.5 | 0 | 6.25 | 5 |

The data indicated that the formulations comprising the specific sweetening agents are higher in flavor quality than the unsweetened formulation (flavor only). Specifically, the sweetened formulations are higher in initial flavor quality, in aftertaste flavor quality with lower total scores for bitterness, throat catch and tongue sting than unsweetened formulation.

### Formulation Example

According to the same manner as described in Example 2, an oily liquid formulation was prepared by mixing with Compound 2 ((−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid), medium-chain triglycerides (USP/NF) and neotame.

### Example 5

### Material and Method

Solubility and sensory tests of sweetening agents were conducted. Specifically, 7.2 mg of neotame, 20 mg of saccharin or 5 mg of sucralose was mixed with medium-chain triglycerides (MCT; USP/NF) to give the total weight of 40 g, 20 g or 100 g, respectively. Additionally, 1 mg of a sweetening agent was mixed with medium-chain triglycerides (MCT; USP/NF) to give the total weight of its mixtures of 100 g. The sweetening agent was sodium saccharin, thaumatin, aspartame, acesulfame potassium, sucrose, glucose, fructose, lactose, D-sorbitol, or xylitol.

Results

Table 5 shows the results of the solubility and sensory tests of the sweeting agents stated above. Table 5 shows that neotame, saccharin and sucralose solved in MCT with the desired sweetness (+), whereas the remaining sweeting agents did not solve in MCT nor show the desired sweetness (−).

TABLE 5

| | Sweetening | Solubility in | Sensory test |
|---|---|---|---|
| | agent | MCT | (sweetness) |
| 1 | Neotame | 0.018 w/w % | + |
| 2 | Saccharin | 0.1 w/w % | + |
| 3 | Sodium | <0.001 w/w % | − |
| | Saccharin | | |
| 4 | Thaumatin | <0.001 w/w % | − |
| 5 | Aspartame | <0.001 w/w % | − |
| 6 | Acesulfame | <0.001 w/w % | − |
| | potassium | | |
| 7 | Sucralose | 0.005 w/w % | + |
| 8 | Sucrose | <0.001 w/w % | − |
| 9 | Glucose | <0.001 w/w % | − |
| 10 | Fructose | <0.001 w/w % | − |
| 11 | Lactose | <0.001 w/w % | − |
| 12 | D-Sorbitol | <0.001 w/w % | − |
| 13 | Xylitol | <0.001 w/w % | − |

What is claimed is:

1. An oral pharmaceutical composition comprising:
(a) a fatty acid derivative represented by the formula (I): 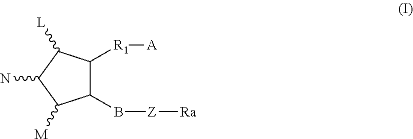
wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;
A is —CH₃, or —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;
Z is 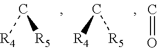
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time;
R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur;
(b) a sweetening agent selected from the group consisting of neotame, saccharin, sucralose, and a mixture thereof; and
(c) a pharmaceutically acceptable oily vehicle.

2. The composition as described in claim 1, wherein Z is C═O.

3. The composition as described in claim 1, wherein B is —CH₂—CH₂—.

4. The composition as described in claim 1, wherein B is —CH₂—CH₂— and Z is C═O.

5. The composition as described in claim 1, wherein L is hydroxy or oxo, M is hydrogen or hydroxy, N is hydrogen, B is —CH₂—CH₂— and Z is C═O.

6. The composition as described in claim 1, wherein the fatty acid derivative is (−)-7-[(2R,4aR,5R,7aR)-2-(1,1-difluoropentyl)-2-hydroxy-6-oxooctahydrocyclopenta[b]pyran-5-yl]heptanoic acid or (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid, its tautomeric isomers thereof or its functional derivative thereof.

7. The composition as described in claim 6, wherein the fatty acid derivative is (−)-7-[(2R,4aR,5R,7aR)-2-(1,1-difluoropentyl)-2-hydroxy-6-oxooctahydrocyclopenta[b]pyran-5-yl]heptanoic acid or (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid.

8. The composition as described in claim 1, wherein the sweetening agent is neotame.

9. The composition as described in claim 1, wherein the oily vehicle is a glyceride.

10. The composition as described in claim 9, wherein said glyceride is a glyceride of a fatty acid having 6-24 carbon atoms.

11. The composition as described in claim 10, wherein said glyceride is a glyceride of a fatty acid having 6-20 carbon atoms.

12. The composition as described in claim 9, wherein said glyceride is a medium chain fatty acid triglyceride.

13. The composition of claim 1, further comprising (d) a flavor.

14. The composition as described in claim 13, wherein said flavor is a vanilla.

15. The composition of claim 1, which is stored in a container.

16. The composition as described in claim 14, wherein said container is a push-pomp type container.
